(19) United States
(12) Patent Application Publication
(10) Pub. No.: US 2026/0165579 A1
(43) Pub. Date: Jun. 18, 2026

(54) METHOD FOR DETECTING A MOVEMENT OF A BODY

(71) Applicant: P3LAB, Ceroux-Mousty (BE)

(72) Inventors: Pierre Martin Jack Gérard DAYE, Braine-l’Alleud (BE); Rémy WOINE, Graux (BE)

(73) Assignee: P3LAB, Ceroux-Mousty (BE)

(21) Appl. No.: 19/122,939

(22) PCT Filed: Oct. 23, 2023
(86) PCT No.: PCT/EP2023/079510

§ 371 Date: Apr. 21, 2025

(30) Foreign Application Priority Data
Oct. 24, 2022 (BE) BE2022/5859

(51) Int. Cl. A61B 3/113; G06T 7/174; G06V 40/18

(52) U.S. Cl. CPC A61B 3/113; G06T 7/174; G06V 40/18; G06T 2207/30041

(57) ABSTRACT

The invention relates to a computer-implemented method for detecting a movement of a body from a sequence of images of this body, by isolating those images (51) belonging to image pairs in which a derivative function (3) of a similarity measure (2) of those image pairs is greater than a predetermined threshold (4). In this way, the movement of the body is detected in these images (51), which can then be processed more precisely to study the movement, without the need to process all the images in the sequence of images.

### TECHNICAL FIELD

The present invention relates to:
a method for detecting a movement of a body,
an eye-tracking method,
an use of the method to detect an eye saccade,
a data processing computer system,
a device for detecting a movement of an eye of a patient,
a computer program, and
a computer-readable medium.

### BACKGROUND

For the purposes of neurological assessment of a patient, it is known to subject the patient's eyes to visual stimuli and to monitor the reaction of the eyes to these stimuli. These stimuli are presented, for example, in the form of one or more pixels on a screen. One or more cameras capture a sequence of images of the patient's eyes simultaneously with the presentation of these stimuli. This sequence of images is then processed to track the movement of the patient's eyes.

The saccades define a known class of movements that can be detected. A “saccade” is a rapid redirection of the line of sight from one centre of interest to another. This is a very rapid eye movement that can reach a speed of 600 degrees per second. In order to detect and track a saccade effectively, the camera's acquisition speed needs to be sufficiently high, for example around 800 frames per second.

The number of images to be processed during this neurological assessment is therefore considerable. Processing them requires high-powered calculation tools and a certain amount of execution time.

In the separate context of the detection of eye blinks, the article “Eye Blink Detection Using Local Binary Patterns” by K. Malik and B. Smolka, ICMCS, IEEE, of 2014, discloses a method for transforming eye images into local binary patterns between which a distance is determined and then regularised. The signal thus obtained comprises peaks that are known to correspond to the eye blinks being sought. Although this method is suitable for a blink of an eye, it does not allow effective detection and tracking of a saccade.

### DISCLOSURE OF THE INVENTION

One of the aims of the invention is to make such treatment more effective. To this end, the invention proposes a method for detecting a movement of a body, for example an eye, from a sequence of images of that body, the method being computer-implemented and comprising the following steps:
(i) determining a similarity measure of each image pair of a sequence of image pairs, the sequence of image pairs being induced by the sequence of images and including each image thereof;
(ii) determining a derivative function of the similarity measure on the sequence of image pairs;
(iii) extracting at least one image from each image pair in which the derivative function is greater than a positive threshold;
(iv) detecting the movement of the body on the basis of the images extracted in the step (iii).

This method enables faster and more efficient detection of movements of the body in the sequence of images, and therefore, ultimately, more efficient processing of these images, particularly when there are a large number of them.

Rather than processing all the images in the sequence for the purpose of detecting a movement of the body, the inventor proposes extracting the images in the sequence that initially contain information about a movement of the body, and detecting this movement in these images. These images can also be processed, for example by segmentation, to track or analyse this movement in greater detail. This means that not all images need to be considered in this respect, which significantly improves the efficiency of the detection of the body movement and, more generally, the processing of images.

One of the reasons for this method, in the context of the prior art, is that approximately 80% of the images captured by the camera do not show any variation in pixels compared with the preceding image in the sequence of images and therefore do not correspond to movements of the eye. To improve the tracking of an eye movement, it was therefore desirable not to process these images, but only those from which an eye movement could be detected, which is what the method according to the invention allows. Of course, this is not limited to this application. The skilled person will understand that it can be used for any detection of movement of a body in a sequence of images. The more the proportion of images on which the motion to be detected is low, the more the method according to the invention is advantageous.

To determine from which images to detect the body movement, and thus which images to extract in the step (iii), the method includes the steps (i) and (ii) that are at the core of the invention.

Firstly, in step (i), a similarity is measured between images. The images are arranged in pairs for this purpose. This measurement is used to determine the degree of similarity between the two images in each pair, typically on a pixel-by-pixel basis. However, this step alone is not sufficient to determine the images to be extracted in step (iii). In fact, two images can show more or less significant variations in pixels without the body represented in the images having undergone any movement. These variations are, for example, due to noise and/or variable luminosity in the body's environment during image acquisition. The step (ii) aims to derive the similarity measure used in the step (i) to eliminate the static component of the images in terms of pixels and better distinguish real body movement from the variations mentioned above. Thus, the extraction of step (iii) is more fine and meets better the efficiency needs sought in this context.

The threshold used in step (iii) can advantageously be used to adapt the proportion of extracted images. It corresponds to a level of compression of the sequence of images. A zero threshold is obviously not desirable, as it would mean keeping all images regardless of their representation. The threshold is set according to the type of similarity measure and derivative function used.

The detection of the body movement can then be detected in step (iv) by processing the extracted images. In one embodiment, the detection takes the form of a simple proof of the existence of a movement of the body that is apparent at first sight from the images from step (iii). The extracted images can be processed in step (iv) to detect and/or characterise the movement of the body more precisely, as is known to the skilled person, the essential point being that this step is carried out on a reduced number of images.

By using the terms “computer-implemented”, the method of the invention involves the use of a computer, a computer network and/or any other programmable (e.g. smartphone, tablet, FPGA, etc.) or programmed (e.g. integrated circuit/ASIC, etc.) device. In particular, the term “computer” cannot be interpreted restrictively.

The determination steps are therefore at least partly based on an underlying computer character. For example, one or more of these steps may consist of a determination by algorithmic calculation.

In the context of this document, the term “body” is used generically and can refer to a physical object as well as all or part of a human body.

For the purposes of this document, the images are preferably the same size, for example 640×480 pixels. However, the method of the invention can be applied selectively to a subset of the pixels in the image, as detailed below. For the purposes of this document, images are preferably captured at the same frequency, for example at 797 frames per second.

The term “sequence” itself implies a notion of order between the elements in the sequence. Preferably, the order associated with the sequence of images is that in which the images were captured, for example by a video camera. For example, in this case, the sequence of images forms a sequence of images over time, representing, for example, a movement of a part of a human body, for example an eye. Other notions of order can be used.

The image pairs from step (i) are also ordered, as they are arranged in sequence. This sequence is “induced” by the sequence of images, i.e. the notion of order in the pairs is induced by the notion of order in the images. In other words, a first pair {I₁, I₂} precedes a second pair {I₁′, I₂′} in the sequence of pairs, with I₁ and I₁′ respectively preceding I₂ and I₂′ in the sequence of images, if and only if, in the sequence of images:
I₁ precedes or corresponds to I₁′ and
I₂ precedes or corresponds to I₂′.
   The images in the same pair are of course preferably separate. All the images in the sequence of images can be found in at least one of the image pairs. The similarity between the images in the sequence of images can thus be measured successively according to the order induced by the sequence of images.

Two preferred examples of sequences of image pairs are provided:
according to a first embodiment, the sequence of image pairs comprises (and preferably consists of) each pair of two consecutive images in the sequence of images;
according to a second embodiment, the sequence of image pairs comprises (and preferably consists of) each pair formed of the first image of the sequence of images and further image of the sequence of images.
   In other words, if the sequence of images corresponds to
(Iₙ)_{1≤n≤N}=(I₁, I₂, I₃, . . . , I_{N}) where N is the number of images said first and second embodiments correspond respectively to the sequences of pairs

$${( \{ {I}_{n} , {I}_{n + 1} \} )}_{1 \leq n \leq N - 1} and {( \{ {I}_{1} , {I}_{n} \} )}_{2 \leq n \leq N}$$

In the first embodiment, the similarity between the images is measured progressively by two consecutive images over the entire sequence of images (image 1 with image 2, then image 2 with image 3, and so on). Thus, it is possible to detect very precisely a lack of similarity between two images that follow each other in the sequence of images. In the second embodiment, the similarity is measured in relation to the first image, so that it is possible to determine from which image a movement of the body could be detected.

The use in this document of the verb “comprise”, its variants or conjugations, does not exclude the presence of elements other than those mentioned. Similarly, the use of the indefinite article “a”, “an”, or the definite article “the” to introduce an element does not exclude the presence of a plurality of these elements.

The use of “similarity measures” to quantify a difference between the pixels of two images is known to those skilled in the art. In particular, there are several such measurements that can be used in the context of the invention, for example by means of a correlation measurement. However, in the context of the invention, an advantageous preferred similarity measure of image pairs is identified. It corresponds to a Bhattacharyya coefficient (denoted “BC”) of two discrete probability distributions associated with the images in the image pair and defined on the basis of pixels in these images.

Specifically, if p and q denote the probability distributions defined over a set P of the pixels of the two images of the image pair, the Bhattacharyya coefficient is defined by:

$$B C ( p , q ) = {\sum }_{x ϵ P} \sqrt{p ( x ) q ( x )} .$$

The set of pixels may correspond to all the pixels in the image or to a subset of them, for example selecting only every other row and/or column. Although it is generally the natural logarithm of the inverse of this coefficient that is used as a similarity measure, in the context of the invention, it is the coefficient itself that is used, particularly for the purposes of step (ii).

There are also several ways known to the skilled person of associating a probability distribution with the pixels of an image in order to evaluate this coefficient. In one embodiment of the invention, the discrete probability distribution p associated with each image is defined at each pixel x of the image by:

$$p ( x ) = \frac{G ( x )}{{\sum }_{y ϵ P} G ( y )}$$

where G is a grey level function defined on the pixels of the image. In other words, p(x) is defined as a quotient of a grey level at pixel p and a sum of grey levels at each pixel in the image. The grey level at a pixel is typically evaluated numerically and discretised, for example on an integer scale ranging from 0 to 255.

Advantageously, the use of the Bhattacharyya coefficient in relation to other similarity measures is independent of the brightness of the images, which makes the method according to this embodiment more robust from step (i), in particular with a view to specifically detecting body movements.

The step (ii) can also be implemented in different ways by means of several discrete derivatives known to the skilled person. In particular, the term “derivative function” is used equivalently to “derivative” in this document, as understood by a person skilled in the art. Given the discrete domain for which the derivative is determined, it is preferably a filtered derivative. In particular, and preferably, a low-pass filter is composed with the derivation of the similarity measure in order to eliminate or reduce any noise on the images. The derivative function is therefore preferably a filtered derivative obtained by combining it with a low-pass filter.

The filtered derivative corresponds preferably to a preferentially bilinear discretization of a transfer function Laplace transform given by H(s)=s (sτ+1)⁻¹ where τ is a constant (more precisely, the time constant of the low-pass filter combined with the derivative). This discretisation allows this regular expression to be used in a digital system. The fact that the discretisation is bilinear is preferred and advantageous in order to guarantee adequate stability of the low-pass filter between its regular form given by the transfer function and its discretised form.

Concretely, if Sₙ corresponds to the similarity measure of the nth image pair of the sequence of image pairs, if S′ₙ corresponds to the evaluation of the derivative function of the similarity measure on this nth image pair, and if t designates a constant duration between two images captured by a camera, the filtered derivative according to this embodiment is defined recursively by

$${S}_{n}^{′} = 2 ( \frac{{S}_{n} - {S}_{n - 1}}{2 τ + t} ) + ( \frac{2 τ - t}{2 τ + t} ) {S}_{n - 1}^{′} .$$

The initial step S′₁=S₁ is typically used. The constant t is a real number, preferably an integer multiple of the sampling period t, this multiple being, for example, between 2 and 10, for example 5.

Returning to the general statement of the method according to the invention, it may be noted that the application (or evaluation) of the derivative function from the similarity measure on the sequence of image pairs naturally provides a data distribution. Preferably, the threshold used in step (iii) is defined by a percentile of this distribution. This is an advantageously practical way of compressing the sequence of images appropriately by extracting a specific percentage of the images according to the purpose of the motion detection. Preferably, this percentile is chosen between the eightieth and the ninety-ninth percentile, which makes image processing significantly more efficient since less than one image in five is extracted from the sequence of images. In the case where the method is aimed at detecting saccadic eye movement, the inventor has found that using the ninety-fifth percentile provides completely satisfactory results, as saccades are very brief and rapid movements. In the particular case of the filtered derivative of the Bhattacharyya coefficient based on a grey level in the pixels of the images, for τ=5t and τ=3.75 ms, it was shown that the corresponding threshold of 0.00175 advantageously made it possible to effectively detect all the saccadic eye movements of any patient, by extracting at most 5% of the images from the sequence of images, whatever the conditions under which the method was carried out.

According to a preferred embodiment of the invention, the image that is extracted from a pair in step (iii) comprises the image with the highest rank in the sequence of images among the two images of the image pair. This allows to keep for step (iv) and/or the possible subsequent processing images in which there is a something happening», in other words, the images where a beginning of movement is likely to be detected. In the case of a pair of the form {Iₙ, Iₙ₊₁}, the image Iₙ₊₁ is therefore extracted, and in the case of a pair of the form {I₁, Iₙ}, the image Iₙ is extracted, if the evaluation of the derivative function from the similarity measure in this pair is greater than the threshold. This embodiment is of course not limitative, and the two images of the pair can optionally be extracted.

The detection of the body's movement can be carried out in step (iv) on the basis of the extracted images alone. However, other images may be used, for example, for reasons of safety or reliability of the method. Thus, according to a preferred embodiment of the invention, the step (iii) also comprises a periodic extraction of an image from the sequence of images. This extraction can be done at a frequency between 20 and 40 Hz, for example 30 Hz, for a usual speed of acquisition of the images by a camera. Alternatively, an image can be extracted every 20 to 40 frames, for example every 30 frames. Given that the proportion of additional images extracted in this way is low, taking these images into account does not significantly affect the efficiency of the method, while at the same time advantageously increasing its reliability and robustness.

Preferably, the method also includes a segmentation step of the images extracted in the step (iii). This segmentation step may take place during the step (iv) and/or simultaneously with or after it. It can form part of further “processing” of the extracted images, for example, to classify, study and/or quantify the body movements detected. The segmentation as such is a method known to those skilled in the art. It can be of any known type. It can, for example, be based on regions or contours within the images in the sequence, and/or also on a classification of pixels by intensity (e.g. luminous, grey level, etc.). The segmentation can be carried out locally, over a defined area of the images, or globally.

Preferably, in the case where the segmentation of each image extracted in the step (iii) is defined by a segmentation function of the image, this segmentation step is implemented via the following sub-steps:
(s1) determining an estimate of the segmentation function of each image extracted in step (iii);
(s2) recursively determining the segmentation function of each image extracted in step (iii) on the basis of its estimate.

This implementation allows the images extracted in the step (iii) to be segmented quickly and efficiently. This is because the segmentation functions are determined recursively, and therefore deduced from each other. The order associated with the recursion is typically that induced by the sequence of images. This allows us to avoid the time-consuming and inefficient processing of individual images. This advantage is further enhanced by the fact that each segmentation function is determined from its estimate (which may or may not be determined recursively, depending on the case), thus constituting an intermediate step that is simpler to perform than direct image segmentation. Fewer calculations are therefore required, as determining a segmentation function is similar to adjusting a residual with respect to its estimate. The recursive aspect also increases the reliability of the determination of image segmentation functions, as this is not based solely on estimates but also on functions that have already been determined.

The term “segmentation function” used above refers to a function defined on the pixels of the images associating a pattern and/or structure of the pixels of the images, typically the pixels having a “non-zero” (or non-constant) image by the function. When such a pattern or structure can be highlighted at the pixel level of a first of the images extracted in step (iii) (either algorithmically or manually), the segmentation step allows this pattern or structure to be advantageously and recursively integrated into the other images extracted in step (iii) in order to monitor and/or analyze its evolution and/or detect it efficiently, without processing each image independently.

The notations (s1) and (s2) should not be interpreted as meaning that the sub-step (s1) is executed in its entirety before the sub-step (s2). These can be interlocked, as in the following preferred embodiment.

Preferably, the estimation of a segmentation function of a second image is determined in the substep (s1) by a composition of a vector field with a segmentation function of a first image, the first image preceding the second image in the sequence of images, the segmentation function of the first image being determined beforehand in the substep (s2), and the vector field corresponding to a displacement of pixels between the second and first images. Preferably, the first image directly precedes the second image in the sequence of images extracted in step (iii). Preferably, each segmentation function estimate of an nth image is determined in a similar way from the segmentation function of the (n−1)th image of the sequence of images extracted in step (iii). Furthermore, the terms “first” and “second” should not be interpreted above as being limited to the position of these images according to the order induced on the images extracted in step (iii) by the order corresponding to the sequence of images.

The vector field corresponds to a form of comparison between two images and encodes a displacement that the pixels of the second image would have to undergo to return to a position associated with the first image. Preferably, it is calculated by optical flow.

The estimates of the segmentation functions, as first-order approximations of these functions, are thus easily calculable, which further improves the efficiency and speed of execution of the segmentation step, and therefore of the method comprising it according to the invention. Furthermore, in the case of images of an eye for eye-tracking purposes, this embodiment allows you to easily deduce a precise estimate of the position of the eye or part of the eye to be tracked on each image extracted in step (iii), and thus greatly limit an area of interest of the image on which to identify the eye or part of the eye via step (s2). The area is all the more limited, and therefore the estimation reliable, if the eye or part of the eye to be tracked varies in position to a limited extent between two consecutive images extracted in the step (iii), which is generally the case when the initial sequence of images comprises a large number of images captured per second. In particular, this design is perfectly suited to the detection and study of saccadic eye movements.

The invention also provides an advantageous eye-tracking method comprising the following steps:
(a) providing a sequence of images of a patient's eye;
(b) detecting an eye movement from the sequence of images;
where step (b) is carried out by means of the method according to the invention. In this case, the method is applied in the case where the “body” corresponds to the eye from the sequence of images provided in step (a). As stated in the method, an eye-tracking is provided on the basis of the detection in step (b).

The method according to the invention enables a more efficient eye-tracking as the eye movement is detected in step (b) more efficiently and quickly. More generally, the embodiments and advantages of the method according to the invention apply mutatis mutandis to this eye-tracking method.

Preferably, step (b) is implemented by means of the method when it includes a segmentation step of the images extracted in step (iii). The aforementioned segmentation step is then preferentially implemented at least in the vicinity of a representation of the iris of the eye on each of the images extracted in step (iii), and the method further comprises the following steps:
(c) determining a position (or position information) of a limbus of the eye on the basis of the segmentation of the images extracted in step (iii);
(d) determining a position (or position information) of the eye on the basis of the position (or position information) of the limbus of the eye determined in step (c).

The method described here allows fast, accurate eye-tracking compared with methods known in the prior art. In fact, the segmentation of the images extracted in step (iii) is carried out recursively, so that each segmentation is deduced from a segmentation estimate and from at least one of the previous segmentations, allowing the segmentation step in step (b) to be carried out quickly and accurately. The segmentation of an image of the eye in the vicinity of the pixels associated with the iris of the eye allows an elliptical shape to be highlighted in the case of contour segmentation, corresponding essentially to the limbus of the eye, the position of which can thus be advantageously deduced in step (c) according to the parameters defining the ellipse.

In addition, the method according to this embodiment proposes to first take account of the limbus to determine the position of the eye in step (d), and thus differs from most known methods which rely distinctively on data relating to the pupil to determine the position of the eye. In fact, determining the position of the eye by these methods is less precise than the method according to the present invention. This is due to the fact that the determination of pupil position characteristics is fraught with several errors due, for example, to the fact that the speed and acceleration of eye movements (which can reach 600°/s and 35,000°/s2 respectively during saccades) induce a relative movement between the pupil and the limbus of the eye, or to the fact that any black spot (e.g. mascara) is taken into account in the calculations by most known eye trackers.

However, the position of the eye can also be determined in step (d) based on the information of the position of the pupil of the eye previously determined in step (c) based on the segmentation of the images extracted in step (iii), allowing even more accurate eye-tracking.

The speed and efficiency of the eye-tracking method also make it particularly interesting for tracking an eye over a continuous stream of video images. The initial sequence of images is then provided in the form of this image stream, for example, by means of a camera pointed at the eye. The subject to which the eye belongs is typically stimulated to follow a moving target on a screen. The eye-tracking method then allows eye movements to be studied, for example, to detect neurodegenerative diseases.

In particular, and in view of the advantages of the invention set out above, the invention specifically proposes a use of the eye-tracking method according to the invention, to detect an eye saccade, typically from a stream of video images of the eye constituting the initial sequence of images.

In general, the applications of the eye-tracking method are not limited to these examples. More generally, the skilled person will understand that the eye-tracking method described herein is only one of several applications of the method described herein. This can be used in a number of practical applications, such as monitoring the eyes of a vehicle driver, facial recognition, for example in video-conferencing software filters, and detecting tumours in images, for example X-rays, via CT and/or CBCT scans.

The present invention also proposes:
a data processing (computer) system comprising means configured to implement the movement detection method according to any one of the embodiments of the invention;
a computer program comprising instructions which, when the computer program is executed by a computer, cause the computer to implement the method according to any one of the embodiments of the invention;
a computer-readable medium (computer) on which the above-mentioned computer program is recorded.

The data processing system comprises, for example, at least one of the following hardware or computer hardware:
a computer equipped with a processor for executing algorithms, preferably with a PC-type architecture or an embedded architecture (ARM);
an ASIC integrated circuit specific to an application and/or an algorithm;
an FPGA-type (logic) integrated circuit, which is typically reconfigurable or reprogrammable after manufacture.

More preferably, the data processing system consists of an embedded system. This embodiment is advantageously made possible because steps (i) to (iii) of the method according to the invention require low computing power, but also and above all because they allow a significant reduction in the number of calculations and computing power required to carry out the subsequent steps of the method, in particular when these include processing the images extracted in step (iii). Said embedded system is typically equipped with an integrated circuit of the FPGA type.

The computer-readable medium preferably consists of at least one computer medium (or set of such media) capable of storing digital information. It comprises, for example, at least one of the following: a digital memory, a server, a USB key or a computer. It can be in a cloud.

The embodiments and advantages of the method according to the invention are transposed mutatis mutandis to the data processing computer system, the computer program and the computer readable medium according to the invention.

The invention also provides a device for detecting a movement of a patient's eye, comprising a viewing chamber for the patient comprising, at a first end, an opening periphery adapted to be in contact with a head part of the patient, and at a second end, a screen for displaying visual stimuli. The screen is integrated into a computer unit comprising:
the computer system according to the invention, typically an embedded system equipped with an integrated circuit of the FPGA type;
at least one camera for acquiring images of the eye directed towards the first end and electronically coupled to the computer system to transmit to the latter a sequence of images of the eye.

The patient typically places his or her head on the opening periphery so as to look into the viewing chamber, and in particular towards the second end where the screen is located. The opening periphery is in contact with the patient's forehead and nose, for example. The viewing chamber thus provides a semi-enclosed environment isolated from external disturbances and suitable for detecting and/or tracking the movement of a patient's eye(s) subjected to visual stimuli, for example for the purposes of neurological assessment. Preferably, the device comprises at least one first such camera directed at a first eye of the patient and at least one second such camera directed at a second eye of the patient to detect and/or track the movement of both eyes of the patient.

As the device comprises the said on-board system, it is made possible to implement the method and/or method according to the invention in real time on the stream of video images acquired by the camera. This embedded technology makes the device according to the invention effective for detecting and/or tracking movement of a patient's eye(s).

The viewing chamber typically comprises a side wall extending around an axis between the first and second ends. Preferably, a distance between the first and second ends is adjustable to a fixed length of that sidewall, measured along the axis, from a variety of possible lengths, these being obtained by successive releasable attachments of one or more removable sidewall portions to a fixed sidewall portion extending from the first end.

This embodiment of the device allows the distance between the first and second ends, i.e. between the patient's eye and the screen, to be modified when the device is in operation, for example, during a neurological assessment of the patient, in order to be able to detect and/or track the movement of the eye under various conditions of distance from the visual stimuli. To modify this distance, it is sufficient to attach or remove one or more of the removable portions, which is particularly easy. Said removable fasteners work in particular like a stack of LEGO® bricks. The fact that each length is fixed allows efficient calibration of the device prior to use, and above all proper use of the device in a standardized environment of known dimensions.

According to one embodiment of the device of the invention, the computer unit further comprises a central clock configured to control:
a display of the visual stimuli, by means of the screen, by refreshing pixels of the screen at a first frequency;
an acquisition of images of the eye, using the camera, at a second frequency; the first and second frequencies being equal to or multiples of each other by a non-zero natural number.

This embodiment, implemented in the computer unit, allows more precise detection and/or tracking of the patient's eye movement. In fact, it's possible to determine exactly which pixel(s) on the screen correspond to an image captured by the camera. There is therefore a temporal certainty in the correspondence of the data provided by the screen and the camera, because the clock with which the computer unit is equipped controls, on its own, the value of the first and second frequencies. In other words, the central clock controls the screen's pixel refresh and the camera's image capture in a synchronized manner. The choice of frequency values as multiple integers of each other allows to maintain the temporal relationship between the acquisition of images by the camera and the presentation of visual stimuli by the screen.

### BRIEF DESCRIPTION OF THE FIGURES

Further characteristics and advantages of the present invention will become apparent from the following detailed description, for the understanding of which reference is made to the appended figures, among which:

FIG. 1 illustrates an example of a graphical representation of the determination of a similarity measure according to step (i) of the method of the invention;

FIG. 2 illustrates an example of a graphical representation of a determination of a derivative function from the similarity measure of FIG. 1 according to step (ii) of the method of the invention;

FIGS. 3A and 3B illustrate two schematic three-dimensional views of a viewing chamber in one embodiment of the device according to the invention;

FIG. 4 illustrates a schematic view of a computer unit in a viewing chamber in one embodiment of the device according to the invention.

The drawings in the figures are not to scale. Similar elements are generally denoted by similar references in the figures. In the scope of this document, the same or similar elements may have the same references. Furthermore, the presence of reference numbers or letters in the drawings cannot be considered as limiting, even when these numbers or letters are indicated in the claims.

### DESCRIPTION OF EMBODIMENTS OF THE INVENTION

This part describes preferred embodiments of the present invention. The latter is described with particular embodiments and references to figures, but the invention is not limited by them. In particular, the drawings or figures described below are only schematic and are not limiting.

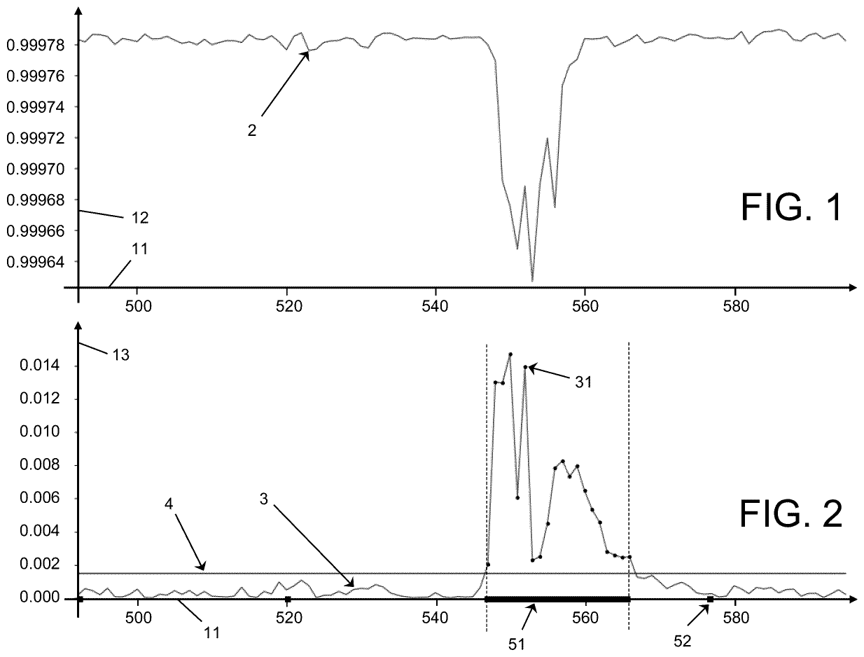

FIGS. 1 and 2 illustrate a concrete example of the execution of steps (i) to (iii) of the method of the invention on a sequence of video images of an eye captured by a camera. The images are captured by the camera at 3.75 ms intervals. They are each associated with a discrete probability distribution defined at a pixel of the image as the ratio between the grey level at this pixel evaluated in a discretised way on a scale from 0 to 255 and the sum of the grey levels at each pixel of the image evaluated in the same way.

The sequence of image pairs considered in performing step (i) consists of each pair formed by two consecutive images in the sequence of images. The similarity measure of each image pair considered is that defined by the Bhattacharyya coefficient (BC) of the two discrete probability distributions associated with the two images of the image pair.

FIG. 1 shows a graph of the evolution of this similarity measure 2. The reference 2 is used to designate both the similarity measure and its graph. The axis 11 indicates the order of the images in the sequence. More precisely, each number corresponds to the number of the image with the highest rank in a image pair, and as the sequence of image pairs is formed by a succession of two consecutive images, numbering the image pairs is the same as numbering the images in the initial sequence. The axis 12 is the one on which the Bhattacharyya coefficient value of each image pair is read.

In order to eliminate the static component of the graph in FIG. 1 and to eliminate the dependence of the Bhattacharyya coefficient on any image noise, a derivative function 3 from the similarity measure 2 on the sequence of image pairs, in accordance with step (ii) of the method, is used, combined with a low-pass filter. More precisely and preferably, a filtered derivative is applied to the similarity measure 2.

FIG. 2 shows a graph of the evolution of such a derivative function 3. The reference 3 is used here to designate both the derivative function and its graph. The filtered derivative considered for this example is more precisely a bilinear discretisation of the transfer function Laplace transform H(s)=s(sT+1)⁻¹ where T is a time constant corresponding to five times the time interval between the capture of two images, i.e. 18.75 ms. This example of a filtered derivative is introduced in the description of the invention, particularly in explicit and discretised form. The value of the derivative function 3 in each image pair can be read on axis 13.

As will be readily apparent to those skilled in the art from FIGS. 1 and 2, applying the derivative function 3 to the similarity measure 2 highlights pairs of consecutive images in which a significant change in terms of pixels has occurred.

The points on the graph of the derivative function 3 are filtered to keep only those 31 with a value read on the axis 13 above a positive threshold 4 as shown in FIG. 2. The threshold of 0.00175 introduced in the description of the invention is used for this purpose. The step (iii) of the method is then performed and corresponds to the extraction of the image 51 with the highest rank among the images of each image pair associated with a point 31 of the graph. On the one hand, this allows a clear eye movement to be detected, approximately between frames 545 and 564, without analyzing the frames one by one. It is thus possible to analyze this movement more precisely by further processing these images 51, for example via the segmentation step and/or steps (c) and (d) of the eye-tracking method described in the presentation of the invention, in particular to detect saccadic eye movements, making such analysis more efficient and rapid and without requiring a great deal of computing power. The method can also be enhanced by the automatic selection, at a certain frequency, for example 30 Hz, of images 52 from the sequence of images which will be considered, along with the images 51, during any subsequent processing.

The method according to the invention can be implemented by a “computer” in the generic sense, for example by an embedded system equipped with an integrated circuit of the FPGA type, which has the advantage of being able to be incorporated into a portable element of limited power, given that the method is particularly efficient. A preferred example of a portable element is shown in FIG. 4. This is a computer unit 7 comprising, in addition to the above-mentioned on-board system, a screen 71, two cameras 72 and two pairs of infrared emitters 73 arranged symmetrically on either side of the screen 71. The computer unit 7 is configured so that the screen 71 displays visual stimuli, for example moving pixels, while the cameras 72 capture images of a patient's eye or eyes. The on-board system can then execute the method according to the invention on the basis of the sequence of images captured by one or both cameras to detect movements of the eye or eyes, for example to detect saccadic eye movements, and optionally process the images extracted from the sequence of images via step (iii), for example, by segmentation as described in detail in the disclosure of the invention.

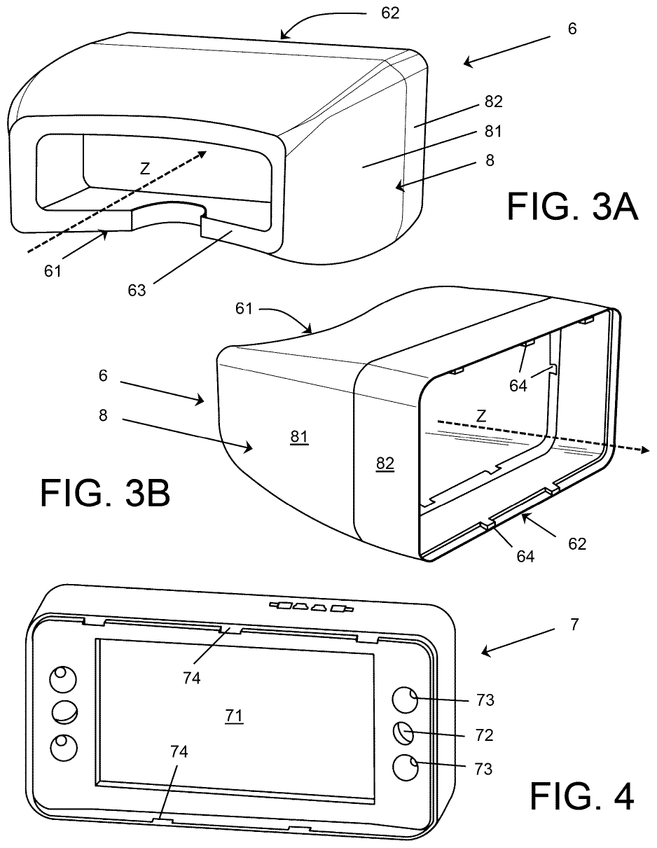

The computer unit 7 is designed to be removably attached to a second end 62 of a viewing chamber 6 shown in FIGS. 3A and 3B. The viewing chamber 6 comprises a side wall 8 of approximately rectangular parallelepiped shape, conically deformed and extending around an axis Z. A first end 61 of the viewing chamber 6 is opposite the second end 62, the axis Z being directed from the first end 61 towards the second end 62. An opening periphery 63 is provided at the first end 61 for contact with a part of the patient's head. When the viewing chamber 6 is in use, the patient's forehead rests against an upper part of the opening periphery 63, and a part of the patient's face between his eyes and mouth, at the level of his nose, rests against a lower part of the opening periphery 63 opposite the upper part, a recess being provided in the lower part for the patient's nose. The patient's eyes are directed towards the inside of the viewing chamber 6, along the axis Z, towards the screen 71 when the computer unit 7 is attached to the second end 62. For the clarity of FIGS. 3A and 3B, the viewing chamber 6 is shown without the computer unit 7.

The viewing chamber 6 provides a calibrated environment suitable for capturing images of the patient's eye(s) by the cameras. The infrared emitters 73 are particularly useful for illuminating the patient's eyes for the purposes of this image capture, given that the environment of the viewing chamber 6 is half-closed and dark. The images captured are therefore preferably infrared images.

The side wall 8 of the viewing chamber 6 consists of a fixed portion 81 and a removable portion 82 which is removably fixed to the fixed portion. To this end, removable fixing elements 64 are provided at the end of the fixed portion 81 opposite the first end 61 and at the ends of the removable portion 82. These fixing elements 64 consist of protrusions and recesses (or more generally reliefs) which cooperate together to establish said removable fixing between the fixed portion 81 and the removable portion 82, in a manner similar to the interlocking of two LEGO® pieces. Advantageously, similar fixing elements 74 are provided on the computer unit 7, for example around the screen 71, to cooperate with the fixing elements 64 of the removable portion 82 at the second end, and thus removably fix the computer unit 7 to the viewing chamber 6. Such a system is very simple and practical.

The removable portion 82 makes the assembly dynamic and enables the distance separating the first end 61, and therefore the patient's eyes, and the screen 71 to be modified. In particular, it offers two fixed, calibrated lengths of side wall 8 measured along the axis Z. A first length is obtained by removing the removable portion 82 and removably fixing the computer unit 7 to the fixed portion 81 via the fixing elements 74 of the computer unit and those 64 at the end of the fixed portion 81. The second length is obtained with the presence of the removable portion 82, as shown in FIGS. 3A and 3B. Of course, other similar removable portions, but optionally not all of the same length measured along the axis Z, can be provided in order to offer more than two possible sidewall lengths 8, i.e. several possible distances between the patient's eyes and the screen. These removable portions 82 can be fixed successively to each other in a removable manner on and from the fixed portion 81, by means of fixing elements 64, thus generalising the assembly of FIGS. 3A and 3B. The computer unit 7 is then positioned at the second end 62, the position of which depends on the number of removable portions 82.

The skilled person will understand that these characteristics on the viewing chamber 6 and its attachment to the computer unit 7 can also be considered independently of the method according to the invention and of the fact that the computer unit 7 comprises means for implementing this method.

In summary, the invention relates to a computer-implemented method for detecting movement of a body from a sequence of images of that body, by isolating (or extracting) the images 51 belonging to image pairs in which a derivative function 3 of a similarity measure 2 of those image pairs is greater than a predetermined threshold 4. In this way, the movement of the body is detected in these images 51, which can then be processed more precisely to study the movement, without having to process all the images in the sequence of images.

The present invention has been described above in relation to specific embodiments, which are purely illustrative and should not be regarded as limiting. It will be readily apparent to the person skilled in the art that the invention is not limited to the examples illustrated or described above, and that its scope is more broadly defined by the claims hereinafter introduced.

1. A method for detecting a movement of a body from a sequence of images of this body, the method being computer-implemented and comprising the following steps:
(i) determining a similarity measure (2) of each image pair of a sequence of image pairs, the sequence of image pairs being induced by the sequence of images and including each image thereof;
(ii) determining a derivative function (3) of the similarity measure (2) on the sequence of image pairs;
(iii) extracting at least one image (51) from each image pair in which the derivative function (3) is greater than a positive threshold (4);
(iv) detecting the movement of the body on the basis of the images extracted in the step (iii).

2. The method according to claim 1, wherein the similarity measure (2) determined in the step (i) corresponds to a Bhattacharyya coefficient of discrete probability distributions associated with the images of the image pair and defined from pixels of these images.

3. The method according to claim 2, wherein the discrete probability distribution associated with each image is defined at a pixel of the image as a quotient of a grey level at that pixel by a sum of grey levels at each pixel of the image.

4. The method according to claim 1, wherein the derivative function (3) determined in the step (ii) corresponds to a filtered derivative obtained by combination with a low-pass filter.

5. The method according to claim 4, wherein the filtered derivative corresponds to a bilinear discretisation of a transfer function Laplace transform H(s)=s(sτ+1)⁻¹ where τ is a constant.

6. The method according to claim 1, wherein the threshold (4) is defined by a percentile of a distribution of data obtained by applying the derivative function (3) to the sequence of image pairs.

7. The method according to claim 6, wherein the percentile is chosen between the eightieth and the ninety-ninth percentile.

8. The method according to claim 1, wherein the step (iii) also comprises periodically extracting an image (52) from the sequence of images.

9. The method according to claim 1, wherein said at least one image (51) extracted in the step (iii) comprises the image of highest rank in the sequence of images among the two images of the image pair.

10. The method according to claim 1, wherein the sequence of image pairs comprises each pair of two consecutive images in the sequence of images.

11. The method according to claim 1, wherein the sequence of image pairs comprises each pair formed of the first image of the sequence of images and a further image of the sequence of images.

12. The method according to claim 1, comprising a segmentation step of the images extracted in the step (iii).

13. The method according to claim 12, wherein the segmentation of each image (51, 52) extracted in the step (iii) is defined by a segmentation function of the image, and wherein the segmentation step is implemented via the following sub-steps:
(s1) determining an estimate of the segmentation function of each image (51 52) extracted in the step (iii);
(s2) recursively determining the segmentation function of each image (51, 52) extracted in the step (iii) on the basis of its estimate.

14. The method according to claim 13, wherein the estimate of a segmentation function of a second image is determined in the substep (s1) by a composition of a vector field with a segmentation function of a first image, and wherein
the first image precedes the second image in the sequence of images,
the segmentation function of the first image is determined beforehand in the sub-step (s2), and
the vector field corresponds to a displacement of pixels between the second and first images.

15. An eye-tracking method comprising the following steps:
(a) providing a sequence of images of an eye of a patient;
(b) detecting a movement of the eye from the sequence of images;
wherein the step (b) is carried out by means of the method according to claim 12.

16. The method according to claim 15, wherein, the segmentation step being implemented at least in the vicinity of a representation of the iris of the eye on each of the images (51, 52) extracted in the step (iii), the method further comprising the following steps:
(c) determining a position of a limbus of the eye on the basis of the segmentation of the images (51, 52) extracted in the step (iii);
(d) determining a position of the eye on the basis of the position of the limbus of the eye determined in the step (c).

17. (canceled)

18. A data processing computer system comprising means configured to implement the method according to claim 1.

19. The computer system according to claim 18, consisting of an embedded system equipped with an FPGA-type integrated circuit.

20. A device for detecting a movement of an eye of a patient comprising a viewing chamber (6) for the patient comprising, at a first end (61), an opening periphery (63) capable of being in contact with a head part of the patient, and at a second end (62), a screen (71) for displaying visual stimuli; the screen (71) being integrated into a computer unit (7) comprising:
the computer system according to claim 19;
at least one camera (72) for acquiring images of the eye directed towards the first end (61) and electronically coupled to the computer system in order to transmit to the latter a sequence of images of the eye.

21. The device according to claim 20, wherein the viewing chamber (6) comprises a side wall (8) extending about an axis (Z), between the first (61) and the second (62) ends, a distance therebetween being adjustable to a fixed length of the side wall (8) measured along the axis (Z) from among different possible lengths, the different possible lengths being obtained by successive removable attachments of one or more removable portions (82) of the side wall (8) to a fixed portion (81) of the side wall (8) extending from the first end (61).

22. The device according to claim 20, wherein the computer unit (7) further comprises a central clock configured to control:
a display of the visual stimuli, by means of the screen (71), by refreshing pixels of the screen (71) at a first frequency;
an acquisition of images of the eye, by means of the camera (72), at a second frequency; the first and second frequencies being equal to or multiples of each other by a non-zero natural number.

23. (canceled)

24. A non-transitory computer-readable medium on which is recorded a computer program comprising instructions which, when the computer program is run by a computer, lead the latter to implement the method according to claim 1.
